(19) United States
(12) Patent Application Publication
(10) Pub. No.: US 2024/0062884 A1
(43) Pub. Date: Feb. 22, 2024

(54) SYSTEMS AND METHODS TO ESTABLISH COMPETENCY FRAMEWORK RELATIONS

(71) Applicant: KONINKLIJKE PHILIPS N.V., Eindhoven (NL)

(72) Inventors: Frank BERGNER, HAMBURG (DE); Paul Anthony SHRUBSOLE, ARNHEM (NL); Milosh STOLIKJ, EINDHOVEN (NL)

(21) Appl. No.: 18/233,378

(22) Filed: Aug. 14, 2023

Related U.S. Application Data
(60) Provisional application No. 63/399,236, filed on Aug. 19, 2022.

(51) Int. Cl. G16H 40/20; G06Q 50/20

(52) U.S. Cl. CPC G16H 40/20; G06Q 50/2057

(57) ABSTRACT

A non-transitory computer readable medium stores at least one database (30) storing clinical competency framework profiles (32) for clinical competencies for a plurality of clinicians at a plurality of medical facilities; and instructions readable and executable by at least one electronic processor (16) to perform a learning activities recommendation method (100) comprising: linking educational content units (38) completed by clinicians to clinical competencies of the clinical competency framework profiles that are fulfilled by the completed learning activities; correlating clinical competency frameworks of different medical facilities that are for the same or similar clinical competencies in the clinical competency framework profiles; and recommending one or more of the educational content units to a clinician seeking to fulfill a clinical competency in one clinical competency framework based on the correlated clinical competency frameworks and the linked educational content units.

### CROSS REFERENCE TO RELATED APPLICATIONS

This application claims the benefit of U.S. Provisional Patent Application No. 63/399,236 filed Aug. 19, 2022. These applications are hereby incorporated by reference herein.

### FIELD

The following relates generally to the medical arts, medical education arts, and medical device operations educational content tracking arts, especially as directed to medical imaging devices.

### BACKGROUND

Complex medical devices offer great flexibility in how they can be used to diagnose, monitor, or treat patients. The performance of the medical device may depend strongly on how the operator uses the device, e.g. setting a non-optimal configuration may provide sub-optimal results whereas using a more optimal configuration may provide better results. Moreover, medical devices that are connected to the Internet or another electronic network may receive software or firmware upgrades over the network that provide new features or enhance existing features; however, these may be useless if the operator is not trained to effectively use the new or enhanced features. Thus, there is substantial benefit to offering education and support to get the best results from the medical devices according to the clinical needs of patients and according to the specializations, way of working of the staff, and the type of hospital or clinical practice.

Health care professionals need to be prepared for unfamiliar situations they might be presented with during their daily practice. To do this, they need to learn how to conduct specific procedures, workflows, protocols, or practices for certain clinical cases/patient-situations that are presented to them.

Skill and competency frameworks describe the skills and knowledge an employee needs to have to fulfill a specific role and job well. Skill and competency frameworks are poorly standardized and specific to clinics, departments, roles and markets. It can be expected that these could include also custom competencies, which might be defined and introduced for them. The problem is that competencies and their meaning are hard to compare, and furthermore it is hard to give recommendations to the learner or human resources (HR) manager for learning activities for those on a system level.

The following discloses certain improvements to overcome these problems and

others.

### SUMMARY

In one aspect, a non-transitory computer readable medium stores at least one database storing clinical competency framework profiles for clinical competencies for a plurality of clinicians at a plurality of medical facilities; and instructions readable and executable by at least one electronic processor to perform a learning activities recommendation method comprising: linking educational content units completed by clinicians to clinical competencies of the clinical competency framework profiles that are fulfilled by the completed learning activities; correlating clinical competency frameworks of different medical facilities that are for the same or similar clinical competencies in the clinical competency framework profiles; and recommending one or more of the educational content units to a clinician seeking to fulfill a clinical competency in one clinical competency framework based on the correlated clinical competency frameworks and the linked educational content units.

In another aspect, a non-transitory computer readable medium stores at least one database storing (i) clinical competency framework profiles for clinical competencies for a plurality of clinicians at a plurality of medical facilities, (ii) educational content units for consumption by the clinicians; and instructions readable and executable by at least one electronic processor to perform a learning activities recommendation method comprising linking educational content units completed by clinicians to clinical competencies of the clinical competency framework profiles that are fulfilled by the completed learning activities; correlating clinical competency frameworks of different medical facilities that are for the same or similar clinical competencies in the clinical competency framework profiles; clustering clinical competency frameworks with similar linked educational content units; and recommending one or more of the clustered educational content units to a clinician seeking to fulfill a clinical competency in one clinical competency framework based on the correlated clinical competency frameworks and the linked educational content units.

In another aspect, a learning activities recommendation method includes linking educational content units completed by clinicians to clinical competencies of clinical competency framework profiles that are fulfilled by the completed learning activities, the clinical competency framework profiles comprising clinical competencies for a plurality of clinicians at a plurality of medical facilities; correlating clinical competency frameworks of different medical facilities that are for the same or similar clinical competencies in the clinical competency framework profiles; determining clinical competencies held by an onboarding clinician at a previous medical facility who is now working at a current medical facility; matching corresponding clinical competency frameworks at the new medical facility with the clinical competency frameworks of the previous medical facility; and recommending the one or more of the educational content units based on the matching.

One advantage resides in providing healthcare professionals with up-to-date skill and competency frameworks.

Another advantage resides in providing recommendation for content for a specific competency that is used in one hospital for a given person, which is difficult as the competency might not be used in other hospitals.

Another advantage resides in providing use-cases in onboarding of staff from a different clinic, assuming that the learning history was logged for instance in a learning-record-store.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for monitoring educational content units in accordance with the present disclosure.

FIG. 2 shows exemplary flow chart operations of the system of FIG. 1.

FIGS. 3 and 4 show correlation data generated by the system of FIG. 1.

### DETAILED DESCRIPTION

Presently, educational content delivery systems can provide a range of learning activities directed to various clinical matters. At the same time, hospitals define clinical competencies in terms of clinical role (e.g. nurse, doctor, radiologist, et cetera) and tasks or classes of tasks that clinicians in that role are competent to perform. Hospitals further define clinical competency frameworks that specify how a clinician in a given role achieves a certain clinical competency. Commonly, the competency framework will include specification that the clinician should complete one or more learning activities (i.e., educational content units) provided via the educational content delivery system.

In general, different hospitals employ different clinical competency frameworks. The framework for a particular type of clinical competency usually has similarities across hospitals due to common regulatory schemes, common considerations of patient safety, and inherent requirements for performing the underlying task or class of tasks. However, different hospitals may define clinical competencies that do not precisely map on one another in scope. For example, one hospital may define a clinical competency for stenting procedures generally, while another hospital may break this into different clinical competencies for cardiac stenting procedures and peripheral stenting procedures. Furthermore, the terminology used in defining a clinical competency can vary between hospitals.

This gives rise to certain problems. In one class of problems, an onboarding clinician who laterally transfers from one hospital to another hospital may have difficulty establishing his or her clinical competencies at the new hospital due to the differing clinical competency frameworks.

In another class of problems, a given hospital establishing a new clinical department or practice has limited guidance in designing the clinical competency framework(s) for that new clinical department or practice. While the hospital might like to adapt a framework from another hospital that already has that clinical department or practice, such adaptation is hindered by differences in frameworks across hospitals.

In another class of problems, two (or more) hospitals with a given clinical department or practice have difficulty in benefitting from cross-pollination of the clinical competency frameworks at the different hospitals. As an example, one hospital may discover that learning activity X is more efficient and/or effective for establishing a given clinical competency than previously used learning activities A and B, and therefore may update its framework by replacing activities A and B with the single activity X. However, due to differences in frameworks, it may not be apparent to other hospitals that this update may be useful for them as well.

A further factor for all these problems is that there is generally no mechanism for cooperation between hospitals in establishing or improving clinical competency frameworks, or for providing guidance in selecting learning activities for establishing clinical competencies for an onboarding clinician.

To address such problems, disclosed herein is a recommender engine for recommending learning activities, or frameworks of learning activities, for various situations. The recommender engine is based on collecting a learning activities database over time.

To construct the learning activities database, the educational content delivery system includes a component provided on a per-hospital basis via which the hospital enters clinical competency framework profiles for its clinical competencies. Each clinical competency framework profile includes a textual description of the clinical competency using the terminology employed at that hospital. Furthermore, each time a clinician at the hospital completes a learning activity, the clinician is given credit by the hospital toward one or more clinical competencies, based on the clinical competency frameworks used by that hospital.

In this way, over time the learning activities database contains a table linking learning activities to clinical competency frameworks on a per-hospital basis. This is done without explicitly defining the competencies, beyond the (possibly brief and inexact) textual descriptions provided by the hospitals.

The learning activities database can be mined by machine learning (ML) to correlate clinical competency frameworks of different hospitals that are for the same or similar clinical competencies. Clinical competency frameworks with similar textual descriptions and similar sets of learning activities can be clustered together to identify similar frameworks, without requiring hospitals to explicitly collaborate with each other.

The resulting machine learned framework groups can be leveraged by the recommendation engine in various ways. For an onboarding clinician, the clinical competency frameworks for clinical competencies held by the onboarding clinician at the old hospital can be matched to corresponding frameworks at the new hospital by clustering to identify clinical competencies the onboarding clinician qualifies for, or almost qualifies for, at the new hospital. The recommender system can then recommend to human resources (HR) the onboarding clinician be recognized for these competencies, along with providing recommendations of any missing learning activities that might be needed to fully qualify for the recommended competencies.

In the case of a new medical department or practice developing a clinical competency framework anew, the hospital can provide a textual description of the contemplated new clinical competency, along with selecting one or two learning activities for the framework. Based on this seed information, the learning activities database can be consulted to identify a cluster of framework groups most closely matching the contemplated new clinical competency, and the learning activities occurring most frequently in that cluster can be recommended to the hospital for inclusion in the newly developing framework. This approach can similarly be used to recommend additional or substitute learning activities to a HR department for updating an existing clinical competency framework.

In yet another use case, the learning activities that most commonly occur in a cluster of clinical competency frameworks can be bundled together as a learning module that is recommended to hospitals as a “core module” for the clinical competency frameworks.

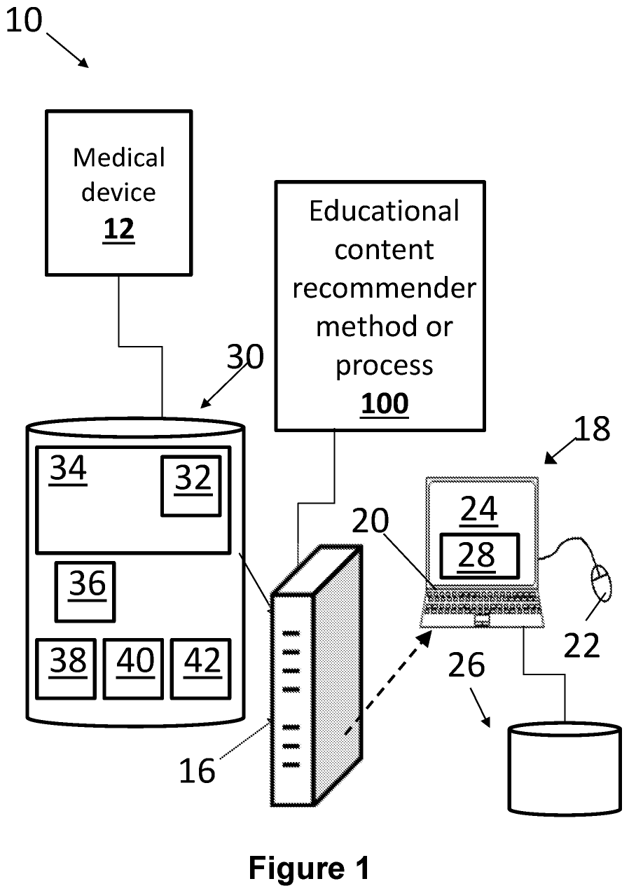

With reference to FIG. 1, an illustrative educational content monitoring system or apparatus 10 for monitoring educational content for a medical procedure employing one or more medical devices 12 (e.g., a medical imaging device 12; or a radiation therapy device; or a combination of the medical imaging device 12 and a biopsy needle, catheter, or other interventional instrument used cooperatively to perform an image guided therapy (IGT) procedure; or so forth). By way of some non-limiting illustrative examples, the medical imaging device 12 may be an interventional X-ray (IXR) or other interventional radiology (IR) system (used in combination with at least one interventional instrument in an IGT procedure), a magnetic resonance imaging (MRI) scanner, a computed tomography (CT) scanner, a positron emission tomography (PET) scanner, a gamma camera for performing single photon emission computed tomography (SPECT), or so forth. As shown in FIG. 1, the educational content generation system 10 includes, or is accessible by, a server computer 16 typically disposed remotely from the medical device(s) 12 used in the medical procedure for which content is to be generated.

FIG. 1 also shows, an electronic processing device 18, such as a workstation computer, a tablet, or more generally a computer. Additionally or alternatively, the electronic processing device 18 can be embodied as a server computer or a plurality of server computers, e.g. interconnected to form a server cluster, cloud computing resource, or so forth. The electronic processing device 18 includes typical components, such as an electronic processor 20 (e.g., a microprocessor), at least one user input device (e.g., a mouse, a keyboard, a trackball, and/or the like) 22, and at least one display device 24 (e.g. an LCD display, plasma display, cathode ray tube display, and/or so forth). In some embodiments, the display device 24 can be a separate component from the workstation 12. The display device 24 may also comprise two or more display devices. The electronic processor 20 is operatively connected with a one or more non-transitory storage media 26. The non-transitory storage media 26 may, by way of nonlimiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the electronic processing device 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the display device 24.

The server computer 16 comprises a computer or other programmable electronic device that includes a non-transitory computer readable medium comprising a database 30 storing clinical competency framework profiles 32 for clinical competencies for a plurality of clinicians at a plurality of medical facilities. The clinical competency framework profiles 32 comprise a textual description of the clinical competency using the terminology employed at the medical facility The clinical competency framework profiles 32 are stored in a table 34 linking learning activities to clinical competency frameworks on a per-medical facility basis.

The database 30 may also comprise multiple databases—for example, the illustrative medical imaging device 12 may generate machine log data as just described that is stored in a machine log database (not shown), and may also generate imaging examination data including images and associated imaging device setting that are stored in a PACS database (not shown).

The database 30 of the server computer 16 can also store a plurality of educational content units 38 for training of clinicians, for example clinicians who operate the device 12. For example, the educational content unit 38 can comprise an animation, a video, and/or a series of images, showing a “best” instance of the procedure, or alternatively an instance of the procedure in which a mistake was made (i.e., to highlight the mistake in the procedure). In a common implementation, the server computer 16 may be a server computer owned or leased or otherwise under the control of the vendor of the medical device 12. In another example, the educational content units 38 are stored in an external server computer (not shown) owned by an entity other than a vendor of the medical device 12.

The database 30 stores instructions executable by the server computer 16 to perform a learning activities recommendation or process 100 implemented by the educational support system 10 for recommending the educational content units 38 for consumption by the clinicians. In some examples, the method 100 may be performed at least in part by cloud processing (that is, the server computer 16 may be implemented as a cloud computing resource comprising an ad hoc network of server computers).

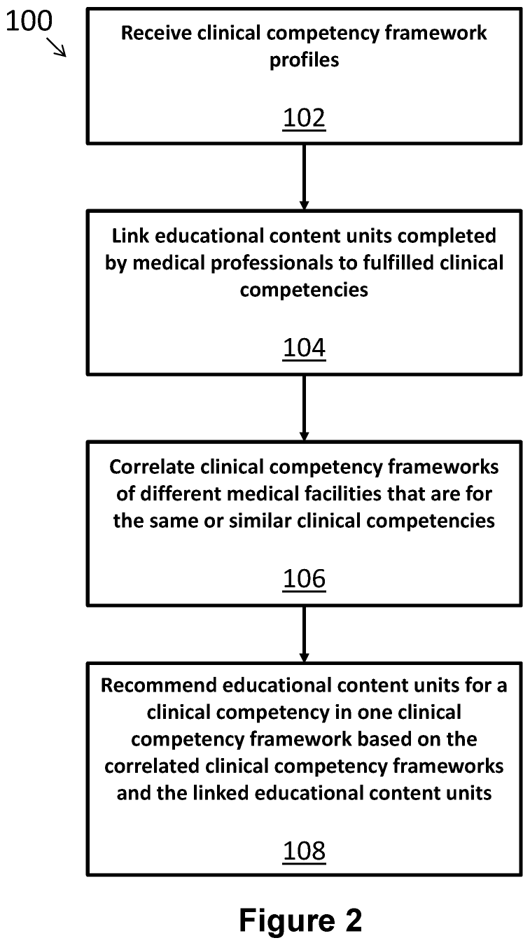

With reference to FIG. 2, and with continuing reference to FIG. 1, an illustrative embodiment of an instance of the method 100 is diagrammatically shown as a flowchart. To begin the method 100, in an operation 102 a representative of each medical facility using the recommender system (e.g. an HR representative) enters clinical competency framework profiles for clinical competencies used at that medical facility. The table 34 of clinical competency framework profiles 32 can be updated based on the information input by the HR representative. Each medical facility enters its clinical competency framework profiles using terminology employed at that medical facility, which may differ from terminology used for similar clinical competencies at other medical facilities. Moreover, the scope of the various clinical competency frameworks of the various medical facilities may differ in various respects. At the stage of operation 102, there is typically no effort to correlate different clinical competency frameworks used at different medical facilities.

In the normal course of operations, clinicians at the various medical facilities complete educational content units 38 as they work toward qualifying for various clinical competencies under the clinical competency frameworks of their respective medical facilities. At an operation 104, educational content units 38 completed by medical professionals are linked to clinical competencies of the clinical competency framework profiles that are fulfilled by the completed learning activities.

At an operation 106, clinical competency frameworks 32 of different medical facilities that were entered at the operation 102 and linked to educational content units fulfilling those competencies in operation 104 are correlated with the same or similar clinical competencies in the clinical competency framework profiles 32. In some embodiments, the correlating operation 102 can be performed by a machine-learning (ML) component 36 implemented in the server computer 16.

In one example of the operation 106, clinical competency frameworks 32 with similar textual descriptions are clustered. In some embodiments, the clinical competency frameworks 32 can also be clustered with similar sets of educational content units 38 to be performed to obtain the clinical competency frameworks 32. Correlated frameworks 32 having similar textual descriptions may also be correlated based on the clustering operation 104. The correlating operation 106 can also include clustering clinical competency frameworks 32 with similar linked educational content units (from operation 104).

At an operation 108, one or more educational content units 38 to be completed by the clinicians are recommended based on the identified frameworks 32 and the linked educational content units from operation 104. In some embodiments, educational content units 38 completed by a clinician can be tracked, and a profile of the clinician can be updated based on the tracked completed educational content units 38.

While FIG. 2 shows a linear flowchart of the operations 102, 104, 106, and 108, it will be appreciated that these various operations may be ongoing to dynamically update the learning activities database. For example, a medical facility may repeat operation 102 at any time to add a new clinical competency framework profile for that medical facility, or to revise a previously entered clinical competency framework profile. Similarly, the operation 104 is ongoing as each time a medical profession completes an educational content unit that is credited to a clinical competency of a clinical competency framework, this adds another link of that educational content unit to the fulfilled medical competency within that framework. The correlation operation 106 may be rerun periodically (e.g., using update clustering) to keep the clinical competency framework correlations current. The recommending operation 108 is repeated each time a recommendation is called for.

The recommending operation 108 can be used in a variety of manners. In one embodiment, clinical competencies held by an onboarding clinician at a previous medical facility who is now working at a current (i.e., new) medical facility can be determined. Frameworks 32 at the new medical facility can be matched with the corresponding frameworks 32 of the previous medical facility. The matching process can include clustering the corresponding frameworks 32 at the new medical facility with the frameworks 32 of the previous medical facility to identify clinical competencies the clinician qualifies for at the current medical facility. One or more additional clinical competencies for the clinician to obtain (i.e., by completing one or more educational content units 38) can be recommended based on the matching. For example, a recommendation can be made to an employee of the current medical facility (i.e., an HR representative) that the clinician be recognized for the clinical competencies held by the clinician at the previous medical facility, and recommending the one or more educational content units 38 to allow the clinician to obtain the additional clinical competencies.

In another embodiment, a textual description of a new clinical competency framework 32 can be received, and a selection of one or more educational content units 38 to be included with the new clinical competency framework 32. To do so, a cluster of frameworks 32 most closely matching the new clinical competency are identified, and one or more educational content units 38 occurring most frequently in the cluster for inclusion in the new clinical competency framework 32 can be recommended. The clinicians can then be required (or receive a recommendation) to complete the educational content units 38 to qualify for the new clinical competency framework 32. In another example, a cluster of clinical competency frameworks 32 most commonly occurring together can be identified, and one or more educational content units 38 for each clinical competency framework in the cluster can be recommended for the clinicians to complete.

### EXAMPLE

The following provides another example of the educational content monitoring system or apparatus 10. Instead of explicitly defining competencies e.g. using taxonomies, here the apparatus 10 uses an implicit representation in a machine-learning model 36. Users (learners, HR managers, and so forth) can pick certain learning activities for their local competency. The apparatus 10 will learn over time if there are competencies in other frameworks 32 with similar activities and therefore can learn a mapping between those. So instead of making recommendations based on user/learning-activity interaction, the recommendations are made based on competency/learning-activity interaction.

It is assumed that the medical facility has established one or more skill/competency frameworks 32 to describe what a certain role should be capable of doing or know to fulfil the given jobs within the clinic or medical facility. Then, the learner or team lead or HR manager of the learner wants to assign learning content to a learner for a given competency, for instance in this hierarchy here: (Role:Nurse)->Clinical Knowledge->Procedures->PCI. A recommendation engine 40 implemented in the server computer 14 can give recommendations and show a list of possible fits for learning content and this given procedure. Assuming that the recommendation does not suffer the cold-start problem, and that potentially already some content was assigned in a way, the recommendation engine 40 now can search for similar content and additionally load the competencies the content was linked to in other frameworks 32.

For example:
Content A (PCI with stent placement: how the stent is placed):
   (Physician)->Clinical Knowledge->Tools->Stenting
   (Physician)->Clinical Knowledge->Tools->Catheter Moving
   (Senior Nurse)->Hybrid OR->Preparations->Backtable Support
Content B . . .

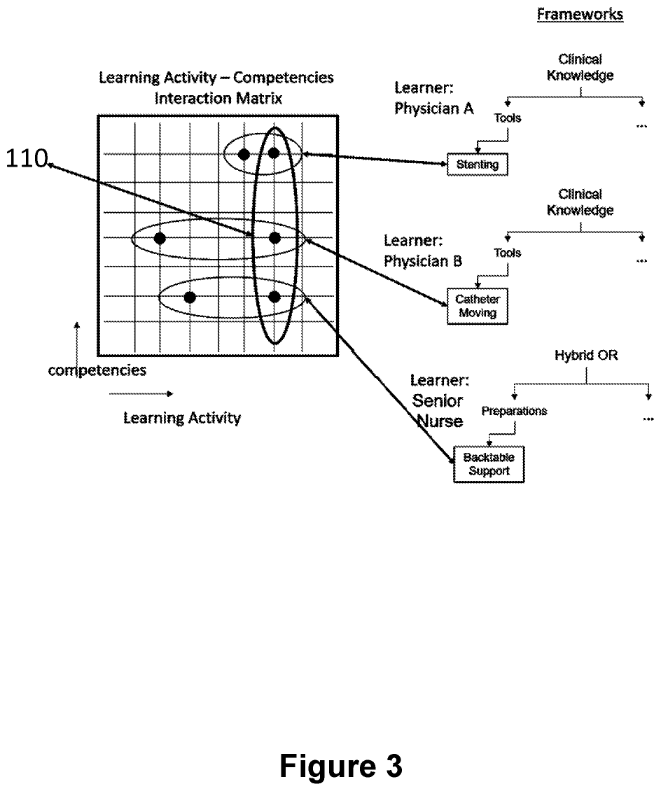

An example of the above content linking is shown in FIG. 3, illustrating production of the recommendation 110. FIG. 3 represents the output of the operation 102 of FIG. 2 by plotting the learning activities database as a grid of competencies (y-axis) versus learning activities (x-axis, i.e. educational content units). FIG. 3 shows that the linkage to the other frameworks 32 gives the user a much richer information in which context and for which competencies a certain learning type was assigned. This information is much richer than what could have been derived from only the abstract of the learning course.

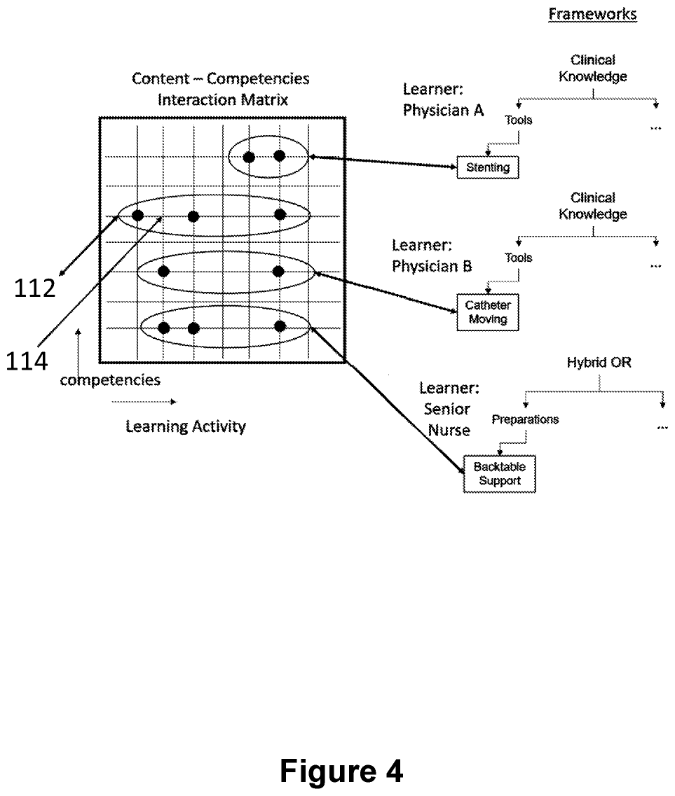

Assuming that the apparatus 10 is operated long enough for the recommender engine 40 to give meaningful results, the GUI 28 now can visualize how certain competencies in different frameworks 32 might interrelate by examining what content was assigned there. This can be useful if new team members are onboarded from a different clinic to also prefill the local competency framework. An example of how the interrelation can look like the in the recommendation model is depicted in FIG. 4, where reference number 112 denotes an output of linked competency frameworks, and reference number 114 denotes an output of recommended educational content units. For the output 112, the nurse has now picked more learning content for the “OR support” competency. As seen in FIG. 4, this has substantial overlap with the “Backtable support” of the Senior Nurse. Although there is no formal mapping between these competencies, the learning content implicitly defines the interrelation. For the output 114, the system can now make recommendations for the nurse for additional learning content (i.e. additional educational content units) taking into account the similarity in competencies between the nurse and the Senior Nurse.

Once the recommender engine 40 has established meaningful competency/learning-activity interactions, then certain competencies can be assigned to users based on the learning activities they performed. The solution is to use the internal model of the recommendation engine 40 to ask for a competency based on collection of learning-activities as input. Based on a similarity measure one can find a ranked list of matching competencies that would be overlap with the given input.

In another embodiment, a reference framework 32 is provided, and we can compare users in this. This can be in particular useful in terms of gamification, so that a user can work towards the goal of fulfilling certain competencies.

In another embodiment, the learner's credentials are taken into account and linked into the skill/competency frameworks. This type of information is then also shown to other users, i.e. for a given content one will also get shown what type of user credentials were linked to the given competencies in the other frameworks. This again will improve the user's decision whether the given content might be applicable for the target learner.

In another embodiment, the content can be freely selected for a given competency by one of more users, which might degrade the estimated relation between competencies and learning activities if too much unspecific content is linked. To prevent this, the apparatus 1 is added an administrative authority that first checks the learning activity and confirms that it can be linked to the given competencies.

In another embodiment, the derived competencies/learning activity relations can be used in a clustering to derive stereotype competencies linked to a common role in a given scope, e.g. a market. As linked learning activities might change over time, the stereotype will be automatically updated over time. From this, changes in necessary competencies can be detected. Another aspect is that the stereotype can be picked to warm-start custom competencies in a clinic, which can be tailored in the next steps to the local needs.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
at least one database storing clinical competency framework profiles for clinical competencies for a plurality of clinicians at a plurality of medical facilities; and
instructions readable and executable by at least one electronic processor to perform a learning activities recommendation method comprising:
linking educational content units completed by clinicians to clinical competencies of the clinical competency framework profiles that are fulfilled by the completed learning activities;
correlating clinical competency frameworks of different medical facilities that are for the same or similar clinical competencies in the clinical competency framework profiles; and
recommending one or more of the educational content units to a clinician seeking to fulfill a clinical competency in one clinical competency framework based on the correlated clinical competency frameworks and the linked educational content units.

2. The non-transitory computer readable medium of claim 1, wherein the correlating includes clustering clinical competency frameworks with similar textual descriptions.

3. The non-transitory computer readable medium of claim 2, wherein the correlating includes identifying frameworks having similar textual descriptions based on the clustering.

4. The non-transitory computer readable medium of claim 1, wherein the at least one database stores the clinical competency framework profiles as a table associating the linked educational content units to the clinical competency frameworks on a per-medical facility basis.

5. The non-transitory computer readable medium of claim 4, wherein the method further includes:
receiving one or more inputs from an employee of the medical facility, the inputs indicative of competency framework profiles for its clinical competencies being entered; and
updating the table based on the received one or more inputs.

6. The non-transitory computer readable medium of claim 4, wherein the clinical competency framework profiles comprise a textual description of the clinical competency using the terminology employed at the medical facility.

7. The non-transitory computer readable medium of claim 1, wherein the at least one database further stores the educational content units for consumption by the clinicians, and the correlating includes:
clustering clinical competency frameworks with similar linked educational content units.

8. The non-transitory computer readable medium of claim 7, wherein the method further includes:
tracking the educational content units completed by a clinician; and
updating a profile of the clinician based on the tracked completed educational content units.

9. The non-transitory computer readable medium of claim 1, wherein correlating clinical competency frameworks of different medical facilities that are for the same or similar clinical competencies in the clinical competency framework profiles includes:
performing the correlating with a machine-learning (ML) component.

10. The non-transitory computer readable medium of claim 1, wherein recommending includes:
determining clinical competencies held by an onboarding clinician at a previous medical facility who is now working at a current medical facility;
matching corresponding clinical competency frameworks at the new medical facility with the clinical competency frameworks of the previous medical facility.

11. The non-transitory computer readable medium of claim 10, wherein matching corresponding clinical competency frameworks at the new medical facility with the clinical competency frameworks of the previous medical facility includes:
clustering the corresponding clinical competency frameworks at the new medical facility with the clinical competency frameworks of the previous medical facility to identify clinical competencies the clinician qualifies for at the current medical facility.

12. The non-transitory computer readable medium of claim 11, wherein recommending one or more additional clinical competencies for the clinician to obtain based on the matching includes:
recommending to an employee of the current medical facility that the clinician be recognized for the clinical competencies held by the clinician at the previous medical facility; and
recommending the one or more educational content units to allow the clinician to obtain the additional clinical competencies.

13. The non-transitory computer readable medium of claim 1, wherein the method further includes:
receiving a textual description of a new clinical competency framework;
receiving a selection of one or more educational content units to be included with the new clinical competency framework.

14. The non-transitory computer readable medium of claim 13, wherein the method further includes:
identifying a cluster of frameworks most closely matching the new clinical competency; and
recommending one or more educational content units occurring most frequently in the cluster for inclusion in the new clinical competency framework.

15. The non-transitory computer readable medium of claim 1, wherein the method further includes:
identifying a cluster of clinical competency frameworks most commonly occurring together; and
recommending one or more educational content units for each clinical competency framework in the cluster.

16. A non-transitory computer readable medium storing:
at least one database storing (i) clinical competency framework profiles for clinical competencies for a plurality of clinicians at a plurality of medical facilities, (ii) educational content units for consumption by the clinicians; and
instructions readable and executable by at least one electronic processor to perform a learning activities recommendation method comprising:
linking educational content units completed by clinicians to clinical competencies of the clinical competency framework profiles that are fulfilled by the completed learning activities;
correlating clinical competency frameworks of different medical facilities that are for the same or similar clinical competencies in the clinical competency framework profiles;
clustering clinical competency frameworks with similar linked educational content units; and
recommending one or more of the clustered educational content units to a clinician seeking to fulfill a clinical competency in one clinical competency framework based on the correlated clinical competency frameworks and the linked educational content units.

17. The non-transitory computer readable medium of claim 16, wherein the at least one database stores the clinical competency framework profiles as a table associating the linked educational content units to the clinical competency frameworks on a per-medical facility basis.

18. The non-transitory computer readable medium of claim 17, wherein the method further includes:
receiving one or more inputs from an employee of the medical facility, the inputs indicative of competency framework profiles for its clinical competencies being entered; and
updating the table based on the received one or more inputs.

19. The non-transitory computer readable medium of claim 15, wherein the method further includes:
tracking the educational content units completed by a clinician; and
updating a profile of the clinician based on the tracked completed educational content units.

20. A learning activities recommendation method, comprising:
linking educational content units completed by clinicians to clinical competencies of clinical competency framework profiles that are fulfilled by the completed learning activities, the clinical competency framework profiles comprising clinical competencies for a plurality of clinicians at a plurality of medical facilities;
correlating clinical competency frameworks of different medical facilities that are for the same or similar clinical competencies in the clinical competency framework profiles;
determining clinical competencies held by an onboarding clinician at a previous medical facility who is now working at a current medical facility;
matching corresponding clinical competency frameworks at the new medical facility with the clinical competency frameworks of the previous medical facility; and
recommending the one or more of the educational content units based on the matching.
